(19) United States
(12) Patent Application Publication
(10) Pub. No.: US 2003/0039990 A1
(43) Pub. Date: Feb. 27, 2003

(54) Non-destructive cell-based assay

(76) Inventor: Eric R. Schuur, Palo Alto, CA (US)

Correspondence Address: MORRISON & FOERSTER LLP, 755 PAGE MILL RD, PALO ALTO, CA, 94304-1018, US

(21) Appl. No.: 10/028,174

(22) Filed: Dec. 20, 2001

Related U.S. Application Data
(60) Provisional application No. 60/140,660, filed on Jun. 23, 1999.

(30) Foreign Application Priority Data
Jun. 23, 2000 (WO) PCT/US00/17365

(51) Int. Cl. C12Q 1/68; C12N 15/85; C12N 5/06

(52) U.S. Cl. 435/6; 435/455; 435/320.1; 435/325

(57) ABSTRACT

The invention provides methods and vector constructs for assaying gene expression. The vector constructs used in the invention comprise a marker which is directed to the cell surface, which in turn can be assayed. By displaying the marker protein at the cell surface, the level of activity of marker gene expression can be assessed without disrupting the cell membrane, also permitting serial measurements.

### CROSS-REFERENCE TO RELATED APPLICATIONS

This application claims priority to PCT/US00/17365, filed Jun. 23, 2000, which claims priority to provisional application No. 60/140,660, filed Jun. 23, 1999, the contents of which are incorporated herein in their entirety.

### STATEMENT OF RIGHTS TO INVENTIONS MADE UNDER FEDERALLY SPONSORED RESEARCH

Work described herein may have been supported by a grant from the U.S. government, which may have rights in this invention.

### TECHNICAL FIELD

The invention relates to cellular gene expression assays, and more particularly to using cell surface markers to detect and/or quantitate intracellular gene expression.

### BACKGROUND ART

Recombinant DNA molecules have been extensively used to direct synthesis within cells of proteins (reporter proteins) that serve as indicators of some biologic process that is a subject of study. These reporter proteins have been designed to provide quantitative data on the subject process with less time, effort and/or cost than previously utilized techniques for measuring the same process. Generally speaking, this is because these systems take advantage of means of detecting the reporter protein that are lower cost, than alternatives for detecting the study process directly. Hence, the study process alters some property of the reporter protein (quantity, activity, and/or spatial distribution), which in turn serves as a surrogate indicator of the state of the study process.

Reporter protein systems have been widely applied to study the regulation of gene expression and illustrate the advantages of reporter protein systems. In this context the property of the reporter protein that is altered is typically the quantity of reporter protein present. Until recently, the most widely used assay procedures for measuring the level of expression of endogenous genes was Northern blot analysis. In this procedure, messenger RNA is obtained from cells by lysing them, purifying the mRNA and analyzing it by electrophoresis and solid phase hybridization to DNA probes corresponding the gene of interest. More recently assay procedures based on amplification of cDNA copies of the mRNA by the polymerase chain reaction (PCR) or hybridization of cDNA to solid phase arrays of known DNA fragments (DNA arrays) have been employed to measure mRNA levels from endogenous genes. Like Northern blot analysis, these procedures require the destruction of cells to measure the level of gene expression.

Recombinant DNA reporter gene systems were developed to enable quantitative, rapid and inexpensive measurement of the activity of the study of transcriptional promoters and enhancers (transcriptional regulatory elements, or TREs) that regulate the transcription of genes. In these procedures the coding regions of a molecularly cloned gene were replaced using recombinant DNA technology by a heterologous DNA sequence termed a reporter gene encoding a reporter protein. This reporter gene directs synthesis of an easily measurable reporter protein. The cloned TRE driving synthesis of the reporter protein was then introduced into cells where, by determining the amount of reporter protein present, the level of activity of the linked TRE(s) under the study conditions could be inferred.

Many different reporter proteins have successfully been used. Usually the protein is not found in the host cell type and the quantity of protein present can conveniently be measured. Recombinant DNAs encoding enzyme are often used as reporter genes due to the sensitivity of enzyme assays. Examples of enzymes used as reporter genes include chloramphenicol acetyltransferase (CAT; Gorman et al., Mol. Cell. Biol. 2:1044 (1982), beta glactosidase (βgal) Jefferson et al., EMBO J. 6:3901 (1987), beta lactamase (βLA) Zlorkamik et al., Science 279:84 (1998), secreted alkaline phosphatase (SEAP; Berger et al, Gene 66:1-10 (1988), beta glucuronidase (GUS) Jefferson et al., EMBO J. 6:3901 (1987) and luciferase (LUC) De Wet et al., Mol. Cell. Biol. 7:725 (1987)). However, in most cases the level of gene expression is inferred by assaying protein in cell extracts, again requiring the destruction of the cells.

For SEAP, βgal and βLA methods have been developed to overcome the barrier of the cell membrane to measure the amount of reporter protein without destroying the cells. As the name suggests, SEAP is secreted into the extracellular medium that can be sampled and assayed for enzymatic activity. This permits serial measurements but requires an assay vessel separate from that containing the cells. Intracellular levels of both βgal and βLA can be measured using synthetic non-fluorescent substrates for the enzymes that can pass through the cell membrane. Once inside the cell the substrate is accessible to the reporter enzyme. Hydrolysis results in a fluorescent product that is trapped intracellularly and can be measured non-destructively, however, loading of the cells can be cumbersome and the quantity of substrate is limited by the small intracellular volume.

Activation of the enzymatic properties of a reporter protein(s) has been used to detect physical binding of macromolecules in the context of live cells. In particular, inactive mutants of βgal, a multisubunit enzyme, are known in the art (Blau et al. PCT WO 98/44350, 1998); certain of these mutants can undergo complementation to form active tetramer holoenzyme when induced to associate. By fusing test protein sequences to two complimenting mutant subunits, then introducing the test protein-βgal fusion genes into a cellular environment where they can be expressed, binding of the test protein sequences can be inferred from the presence of βgal activity in the cells.

Alterations in the spatial distribution of a reporter protein is an additional way to use reporter proteins as indicators of an intracellular process. Subcellular distribution of proteins is directed by elements of their primary amino acid sequence termed topogenic sequences (Palade 1975; Wickner and Lodish 1985; van Geest and Lolkema 2000). Such topogenic sequences that can direct proteins to localize in the nucleus, mitochondria, secretory vesicles, cell membrane or other cellular compartments are well known in the art. In the absence of a topogenic sequence proteins are freed into the cytoplasm.

Nuclear localization signals, such as that found in the SV40 virus large T antigen, have been used to direct the nuclear localization of βgal in certain reporter protein systems (Kalderon, Roberts et al. 1984). The resulting distribution of βgal both concentrates the enzyme to produce a more intense signal and shifts the signal to a different subcellular location than that of endogenous βgal enzymes, enabling more convenient assessment of βgal expression during immunohistochemistry procedures.

Topogenic sequences that direct transport of proteins to the cell surface have been extensively studied, but have not been used as a component of a reporter system. Primarily, fusion proteins of signal sequence (SS), transmembrane domain (TMD) and stop-transfer sequence (STS) with heterologous sequences have been used to study the function of the topogenic sequences themselves, with the heterologous sequences serving primarily as a neutral background with respect to the protein transport process, although U.S. Pat. No. 5,866,344 discloses methods utilizing an SS and TMD in conjunction with sequence encoding a recombinant antibody for isolating recombinant antibodies which are expressed on the surface of host cells. Kundu et al. (Hiebert and Lamb 1988; Kundu, Jabbar et al. 1991) fused the amino terminal SS and TMD of influenza virus neuraminidase (NA) to the ectoplasmic domain of the transferrin receptor to examine NA sequences required for transport and oligomerization. Similarly, Heibert and Lamb (Hiebert and Lamb 1988; Kundu, Jabbar et al. 1991) fused the amino terminal SS and TMD of SV5 virus HN protein to pyruvate kinase (a cytoplasmic enzyme) to investigate the HN sequences required for cell surface transport.

### DISCLOSURE OF THE INVENTION

The invention provides methods and compositions for detecting and assaying gene expression in living cells through measurement of marker proteins displayed at the cell surface. In addition, methods for assaying the marker protein to quantitate intracellular gene expression are provided. The marker proteins described herein can be assayed by simple addition of a detection reagent (e.g., a colorimetric substrate when the marker is an enzyme, or a labeled reagent which binds specifically to the marker) to the cultures of cells. Accordingly, the present invention provides advantages relative to other reporter systems currently in use by removing the barrier of the cell membrane, allowing substrates and other indicator molecules to directly and immediately contact the marker protein. Furthermore, through use of two or more cell surface displayed marker proteins with different substrates or indicator molecule specificities, alterations in expression of multiple genes can be assessed in parallel and in real time.

The invention also provides methods of purification of cells expressing a cell surface marker. Thus, an additional advantage of the present invention is the ability to use multiple modes of purification of cells expressing the marker proteins, surmounting the limitation of presently practiced methods that rely on use of expensive fluorescence activated cell sorting equipment.

The invention provides vectors which are useful for expressing a marker on a cell surface, for detecting gene expression using a reporter gene construct, for quantitating expression of a marker or marker gene sequence in a host cell, and methods for detecting and/or quantitating activity of a TRE and methods for quantitating expression of a marker or marker gene sequence in a host cell. Vectors used in the methods of the invention comprise a nucleotide sequence encoding a fusion polypeptide, said fusion polypeptide comprising (a) a signal sequence; (b) a membrane attachment moiety; and (c) a marker, wherein said signal sequence, membrane attachment moiety and marker are operably linked in frame and wherein the vector lacks a transcriptional regulatory element (TRE) operably linked with said nucleotide sequence. The membrane attachment moiety may or may not be heterologous with respect to the marker. In certain embodiments, the membrane attachment moiety is a transmembrane domain.

Vectors may further comprise a multiple cloning site and/or a nucleotide sequence for selection in mammalian cells.

The marker may be an enzyme, such as a restriction endonuclease, or a proteinaceous member of a binding pair, such as a epitope. The marker may also be a domain, subunit, or fragment of an enzyme or proteinaceous member of a binding pair.

The invention also provides host cells comprising a vector as described. The host cell may be, for example, a mammalian cell, such as a mammalian tissue culture cell line or primary cells.

Also provided are kits comprising a vector as described. The kits may additionally comprise a detection reagent for detecting the marker.

The invention further provides methods for detecting expression of a reporter gene construct in a host cell, by detecting a marker encoded by the reporter gene construct, wherein the marker is associated with the cell surface, and wherein said reporter gene construct comprises a nucleotide sequence encoding a fusion polypeptide comprising a signal sequence, a membrane attachment moiety and a marker, said membrane attachment moiety heterologous to said marker, and wherein said signal sequence, membrane attachment moiety and marker are operably linked in frame, and wherein said nucleotide sequence is operably linked to a transcriptional response element (TRE) which is functional in the host cell.

The TRE may be endogenous or heterologous with respect to the host cell, and the marker may not be naturally associated with the cell surface of the host cell. The marker may be an enzyme, such as a restriction endonuclease, or a proteinaceous member of a binding pair, such as a epitope. The marker may also be a domain, subunit, or fragment of an enzyme or proteinaceous member of a binding pair.

The reporter gene construct (or a vector comprising the reporter gene construct(s)) may be extrachromosomal or integrated into a chromosome of the host cell.

The invention also provides methods for isolating a cell which expresses a marker on its surface, where the marker is expressed from a reporter gene construct comprising a nucleotide sequence encoding a fusion polypeptide comprising a signal sequence, a membrane attachment moiety and a marker, and wherein said signal sequence, membrane attachment moiety and marker are operably linked in frame, and wherein said nucleotide sequence is operably linked to a transcriptional response element (TRE) which is functional in said host cell, by binding the marker to a binding partner which specifically binds to the marker to form a complex between the binding partner and the marker on the cell surface; and isolating the cells which contain the complex. The membrane attachment moiety may or may not be heterologous with respect to said marker. The TRE is generally heterologous with respect to the marker nucleotide sequence (i.e., is not naturally associated with the marker sequence).

Further provided are methods for detecting expression of a reporter gene construct encoding a marker which is associated with the cell surface, by binding a binding partner to the marker, wherein said binding partner which specifically binds to the marker to form a complex between the binding partner and the marker on the cell surface; and isolating the cells which contain the complex, wherein said marker is expressed from a reporter gene construct integrated into a chromosome of the host cell, said reporter gene construct comprising a nucleotide sequence encoding a fusion polypeptide comprising a signal sequence, a membrane attachment moiety and a marker, and wherein said signal sequence, membrane attachment moiety and marker are operably linked in frame, and wherein said nucleotide sequence is operably linked to a transcriptional response element (TRE) which is functional in said host cell. The membrane attachment moiety may or may not be heterologous with respect to said marker. The TRE is generally heterologous with respect to the marker nucleotide sequence (i.e., is not naturally associated with the marker sequence).

The invention also provides methods for detecting expression of a reporter gene construct in a host cell, comprising:

detecting a marker encoded by the reporter gene construct, wherein the marker is associated with the cell surface, and wherein said reporter gene construct comprises a nucleotide sequence encoding a fusion polypeptide comprising a signal sequence, a membrane attachment moiety and a marker, and wherein said signal sequence, membrane attachment moiety and marker are operably linked in frame, and wherein said nucleotide sequence is operably linked to a transcriptional response element (TRE) which is functional in said host cell and wherein the TRE is heterologous with respect to the marker. In some embodiments, the membrane attachment moiety is heterologous with respect to the marker. In some embodiments, the membrane attachment moiety is a transmembrane domain, which may or may not be heterologous with respect to the marker.

These and other aspects of the invention are made apparent to those skilled in the art by the disclosure below. All references cited herein are incorporated by reference in their entirety.

### BRIEF DESCRIPTION OF THE DRAWINGS

FIG. 1: Firefly luciferase reporter gene vector. Top vector of FIG. 1 diagrams the structure of pNALuc, a vector containing a fusion gene of the amino terminal segment of influenza virus NA and the Photinus pyralis luciferase gene. Bottom vector of FIG. 1 diagrams pCMV-NALuc, which is pNALuc with the CMV IE promoter/enhancer driving NALuc transcription.

FIG. 2: DpnI endonuclease expressed at the cell surface. FIG. 2 diagrams construction of vectors used to express DpnI restriction endonuclease from S. pneumoniae on the outer surface of mammalian cells.

FIG. 3: Fluorescent substrates for nucleases. FIG. 3 is a diagram of a substrate for fluorescent assay of cell-surface DpnI molecules. The top strand contains the DpnI recognition sequence embedded in a 10 base oligonucleotide. The complimentary bottom strand is diagrammed below. The notation “mA” denotes methylated adenine residues) the circled R represents a fluorescent molecule covalently linked to the 5′ end of the upper strand while the circled Q represents a quencher molecule covalently linked to the 3′ end of the molecule.

FIG. 4: Fluorescence assay for DpnII restriction endonuclease activity. Each panel shows the level of fluorescence (in arbitrary units) from the substrate over time for reactions containing various enzymes. The approximate assay time was 60 minutes. Panel A: no enzyme; panel B: DNAseI; panel C: EcoRI; panel D: DpnII.

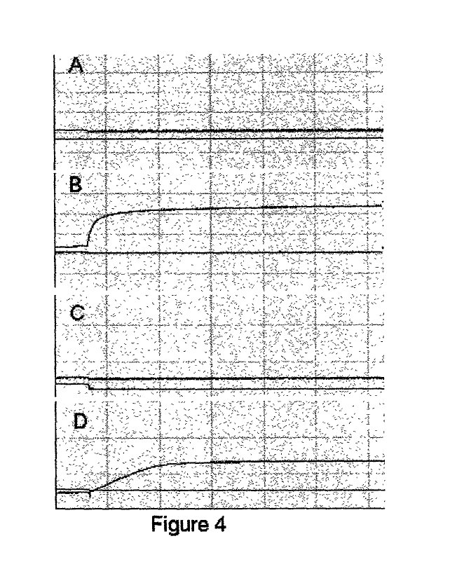

FIG. 5: Bacterial luciferase expressed at the cell surface. FIG. 5 diagrams construction of vectors used to express luciferase from V. harveyi on the outer surface of mammalian cells.

### MODES FOR CARRYING OUT THE INVENTION

The invention provides methods of and compositions for detecting and/or quantitating intracellular expression of a marker gene. The methods of the invention utilize a reporter gene construct operably linked to a transcriptional response element (TRE), where the reporter gene construct encodes a marker, a membrane attachment moiety which may or may not be heterologous with respect to the marker, and a signal sequence, where the marker, heterologous membrane attachment moiety and signal sequence are linked in frame. Upon expression of the reporter gene construct, the marker is displayed on the extracellular surface of the cell. Display of the marker at the cell surface allows measurement of the activity of the linked TRE (as well as indication of events downstream from transcription) without disrupting the cell membrane, thus permitting serial measurements. Quantitation of the cell surface marker encoded by the reporter gene construct allows for quantitative measurements of TRE activity.

In certain embodiments, an enzyme is used as the marker, increasing sensitivity of the assays. An additional advantage of displaying the marker enzyme on the cell surface is that a greater quantity of substrate will be accessible to the enzyme, increasing the amount of product that can accumulate before substrate becomes limiting in the assay. This in turn will result in a wider dynamic range for measurements of gene expression.

Additional benefits to displaying a marker protein at the cell surface relate to purification of the cells expressing the marker protein. Unlike intracellular or secreted maker proteins (e.g., βLA or SEAP), directing a marker to the cell surface provides a physically accessible recognition point on cells expressing the reporter gene construct but not on cells lacking reporter gene expression (or undectectable levels of expression). Therefore, options for physical purification of cells expressing the reporter protein are increased. For example the marker displayed on the cell surface can be bound by a ligand (such as an antibody) which will enable purification of cells expressing the reporter by fluorescence activated cell sorting (Current Protocols in Immunology, Ch. 5. Eds-Coligan et al. John Wiley & Sons, N.Y.). In addition, cells expressing the marker can be physically separated from non-expressing cells through interaction with ligand immobilized on a solid substrate (see, e.g., Greenberg et al., 1985, Blood 65:190).

Cell surface displayed markers can be used to measure the activity of endogenous genes (e.g., genes marked through gene trapping) or the activity of recombinant plasmids containing cloned whole or partial genes. The most immediate use will be to measure the level of activity of promoters and enhancers driving expression of a reporter gene construct. However, this approach should be applicable to measuring regulatory events at the post-transcriptional or translational levels or any event that proceeds display of the marker protein at the cell surface.

General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987, and periodic updates); “PCR: The Polymerase Chain Reaction”, (Mullis et al., eds., 1994); and “Current Protocols in Immunology” (J. E. Coligan et al., eds., 1991).

Definitions

A “transcriptional response element” or “transcriptional regulatory element” or “TRE” is a polynucleotide sequence, preferably a DNA sequence, which regulates transcription of an operably linked polynucleotide sequence in a host cell that allows that TRE to function. A TRE can comprise an enhancer, a promoter, a silencer, an operator, and the like.

A TRE which is “endogenous” to a cell or with respect to a cell is a TRE which is naturally-occurring in that cell. Reference to “a” cell includes any other cell of the same general origin. For instance, a TRE which is endogenous to a COS cell is a TRE which naturally occurs in any COS cell, not just the cell into which the TRE is being used.

A “marker gene”, “marker sequence”, “reporter gene” or “reporter sequence”, used interchangeably herein, refers to a polynucleotide sequence whose expression product, or marker, (whether transcription and/or translation) can be detected by methods known in the art and described herein. Detection may be by any means, including but not limited to visible to the naked eye, spectroscopic, photochemical, biochemical, immunochemical, or chemical means.

A “marker” is an expression product of a marker gene, and includes enzymes and proteinaceous members of specific binding pairs. The term “marker” can be an intact polypeptide (such as a receptor or holoenzyme) or a region, or portion, of a polypeptide. Examples of such regions include, but are not limited to, a ligand binding region of a receptor, a catalytic domain of an enzyme, a portion of an enzyme which binds a ligand, an epitope of a polypeptide.

A “binding pair” or “specific binding pair” is a pair of molecules which specifically bind to one another. Examples of specific binding pairs include receptors and their cognate ligands, antibodies and their cognate antigens and lectins and their cognate carbohydrates. A “proteinaceous member of a binding pair” refers to a polypeptide-comprising member of a binding pair. For example, a receptor is a proteinaceous member of the binding pair, and the receptor ligand is the binding partner of a binding pair comprising a receptor and its cognate ligand.

As used herein, the term “vector” refers to a polynucleotide construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, or a “viral vector” which is designed to result in the production of a recombinant virus or virus-like particle, or “shuttle vectors”, which comprise the attributes of more than one type of vector.

A “membrane attachment moiety” is a moiety which effects association of a marker with a cell membrane. The nature of the association may be on the surface of the cell membrane or inserted, or integrated, into the cell membrane. Examples of membrane attachment moieties are provided herein and include, but are not limited to, transmembrane domains. A membrane attachment moiety may be of any chemical composition, including polypeptide, lipopolypeptide, glycosylated polypeptide, a combination of glycosylated and lipopolypeptide, and glycolipid.

A first moiety which is “heterologous” with respect to a second moiety is not naturally associated with the second moiety. For example, a marker which is “heterologous” with respect to the cell surface is not naturally found on the cell surface. A TRE which is “heterologous” with respect to a marker is not naturally associated with that marker.

A membrane attachment moiety (such as a transmembrane domain) which is “heterologous” with respect to a marker is not naturally associated with the marker with which it is being associated as a fusion polypeptide. Conversely, a marker which is heterologous to a membrane attachment moiety is not naturally associated with the membrane attachment moiety with which it is being associated as a fusion polypeptide. For example, the transmembrane domain of the vesicular stomatitis virus G protein is heterologous to markers which are not derived from the vesicular stomatitis virus G protein.

“Under transcriptional control” is a term well understood in the art and indicates that transcription of a polynucleotide sequence, usually a DNA sequence, depends on its being operably (operatively) linked to an element which contributes to the initiation of, or promotes, transcription.

“Operably linked” refers to a juxtaposition wherein the elements are in an arrangement allowing them to function.

A “gene” refers to a coding region of a polynucleotide. A “gene” may or may not include non-coding sequences and/or regulatory elements.

A “host cell” includes an individual cell or cell culture which can be or has been a recipient of the vector(s) described herein. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a vector described herein.

A “fusion polypeptide” is a polypeptide comprising regions in a different position in the sequence than occurs in nature. The regions normally exist in separate proteins and are brought together in the fusion polypeptide; or they may normally exist in the same protein but are placed in a new arrangement in the fusion polypeptide. The fusion of regions may be direct (i.e., directly abutting) or separated by one or more amino acids (i.e., indirect due to intervening sequences). For purposes of this invention, the fusion polypeptide, when expressed in a suitable host cell, becomes associated with the membrane, or surface, of the host cell.

“Expression” includes transcription and/or translation.

As used herein, the term “comprising” and its cognates are used in their inclusive sense; that is, equivalent to the term “including” and its corresponding cognates.

“A,” “an” and “the” include plural references unless the context clearly dictates otherwise.

Conditions that “allow” or “permit” an event to occur, such as expression, or formation of a complex between a marker and a binding partner or ligand, are conditions that do not prevent such events from occurring. Thus, these conditions permit, enhance, facilitate, and/or are conducive to the event, such as binding of an antibody to an antigen. Such conditions, known in the art and described herein, depend upon the nature of the marker as well as the binding partner (i.e., degree of binding specificity). These conditions also depend on what event is desired, such as detection, expression or infection.

“Expressing a marker on a cell surface”, “displaying a marker on the cell surface”, and the like, mean that the marker is associated with the cell membrane and is accessible to react with a binding partner and/or substrate which is contacted with the cell surface.

A “signal sequence” is an amino acid sequence that directs nascent and newly synthesized polypetides into the export compartment of a cell (e.g., into the endoplasmic reticulum in higher eukaryotic cells). As is known in the art, signal sequences are short, typically about 15 to 25 amino acids in length, generally hydrophobic sequences that are usually located in the N-terminal portion of a polypeptide and are generally cleaved after the polypeptide has crossed into the export compartment. As will be apparent to one of skill in the art, a signal sequence may be encompassed within a transmembrane domain.

A binding partner that “specifically binds” its cognate binding partner binds or associates more frequently, rapidly, with greater duration and/or with greater affinity to its cognate binding partner as compared to other moieties.

A “stable complex” formed between any two or more components refers to a complex that is sufficiently long-lasting to persist between its formation and its detection, including any optional washing steps or other manipulation(s) that may take place before detection.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxynucleoside phosphoramidate (P—NH2) or a mixed phosphoramidate-phosphodiester oligomer. Peyrottes et al. (1996) Nucleic Acids Res. 24: 1841-8; Chaturvedi et al. (1996) Nucleic Acids Res. 24: 2318-23; and Schultz et al. (1996) Nucleic Acids Res. 24: 2966-73. A phosphorothiate linkage can be used in place of a phosphodiester linkage. Braun et al. (1988) J. Immunol. 141: 2084-9; and Latimer et al. (1995) Mol. Immunol. 32: 1057-1064. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

Reporter Gene Constructs

The methods and compositions of the invention utilize a reporter gene construct(s). The reporter gene construct comprises a polynucleotide sequence encoding a fusion polypeptide comprising a marker, a membrane attachment moiety which is generally but not necessarily heterologous with respect to the marker, and a signal sequence. In some embodiments, the signal sequence is incorporated within the membrane attachment moiety. The reporter gene construct can be constructed from any naturally occurring or synthetic polynucleotide sequence that encodes the appropriate amino acid sequences using methods (such as recombinant methods) well known in the art.

The marker encoded by the reporter gene construct may be any detectable protein. Preferred markers include enzymes and proteinaceous members of specific binding pairs. The marker is heterologous to the surface of the host cells (i.e., it is not naturally found on the surface of the host cells, although the marker may be an intracellular protein naturally found in the host cell). The marker can be an intact polypeptide (such as a receptor or holoenzyme) or a region, domain, portion, or fragment of a polypeptide. Examples of such regions include, but are not limited to, a ligand binding region of a receptor, a catalytic domain of an enzyme, particularly an active catalytic domain of an enzyme, a portion of an enzyme which binds a ligand, an epitope of a polypeptide, and the like.

In a preferred embodiment the marker is or comprises an active enzyme molecule not ordinarily or naturally found on the extracellular surface (i.e., is heterologous with respect to the cell surface). One example of a heterologous cell surface displayed enzyme is influenza neuraminidase (Bos et al., PNAS 81:2327 (1984)). As noted above, the enzyme does not have to be completely heterologous with respect to the cell but only with respect to the physical location. For example, pyruvate kinase can be displayed at the cell surface (Heibert and Lam, J. Cell Biol. 107:865 (1988)); if it is the only pyruvate kinase activity extracellularly then it can serve as a reporter of gene expression. Alternatively, examples of enzymes heterologous with respect to the cell include βgal, βLA and restriction endonucleases. For any of these the coding region of the enzyme gene can be modified by standard recombinant techniques to incorporate the appropriate topogenic sequences to direct expression of the enzyme to the cell surface.

Alternatively, the marker may be non-enzymatic and serve as, for example, a receptor for ligands, or be any other proteinaceous member of a binding pair designed to enable identification or quantitation of the reporter protein on the cell surface. The marker could serve as an attachment point for ligands that can be detected by physical means (e.g., fluorescent ligands) or for ligand that can be detected by chemical means (e.g., ligand coupled to enzymes). Examples of non-enzymatic extracellular domains include the extracellular domain of VSV-G protein (which can be recognized by antibody specific for VSV-G) or avidin (in combination with its ligand, biotin).

Any naturally-occurring or synthetic nucleotide sequence(s) which encodes amino acid sequences which are capable of functioning as a signal sequence and/or membrane attachment moiety may be used in constructing the reporter gene construct. Generally, the membrane attachment moiety sequence is heterologous to the marker (at both the nucleotide and amino acid sequence levels). Any sequences which direct translated protein into the intracellular pathways used to process integral membrane proteins (Wickner and Lodish, Science 230 :400 (1985); (Hegde and Lingappa 1997)) may be used. Genes encoding integral membrane proteins can serve as a source of DNA sequences encoding protein domains that will localize on the outer surface of the cell. Examples of integral membrane proteins found in eukaryotic cells include vesicular stomatitis virus G protein, low density lipoprotein receptor, the human transferrin receptor and influenza neuraminidase. Prokaryotic cells synthesize integral membrane proteins as well (Georgiou et al., U.S. Pat. No. 5,348,867) which may be used. Examples of such proteins are OmpA (Stathopoulos et al., Appl. Microbiol. Biotechnol. 45:112 (1996)) and OmpT coli (Stumpe et al., J. Bacteriol. 180:4002 (1998)) of Escherichia.

Preferably, nucleotide sequences encoding topgenic sequences derived from naturally-occurring polypeptides are fused to heterologous reporter gene sequences in a similar configuration to the integral membrane protein from which they were derived. For example, the amino terminal 48 amino acids of influenza virus neuraminidase can be fused to the amino terminal region of the transferrin receptor to provide the necessary signal sequence and transmembrane domain functions to localize the fusion protein to the cell surface (Kundu et al., Mol. Cell Biol. 11:2675, 1991). In a second example, the transmembrane domain of CD3-epsilon is inserted in the carboxy terminal portion of the rat growth hormone gene, similar to its natural location (Alonso and Alarcon, J. Biol. Chem. 272:30748, 1997). The resulting fusion protein is efficiently directed to the cell surface.

Generally, two types of membrane attachment moieties can direct proteins to the surface of the cell. The most extensively studied comprise one or more transmembrane domains (TMD), which may occur one or more times throughout the protein sequence (Wickner and Lodish 1985; van Geest and Lolkema 2000). The TMD may include a signal sequence (SS) (Brown, Hogue et al. 1988). The TMD(s) embed within the lipid bilayer of the membrane during translocation into the endoplasmic reticulum, anchoring the protein to the membrane. In addition, a stop transfer signal (STS) may be present. Depending on the organization of the above topologic sequences, many different integral membrane protein topologies can be achieved: when the TMD is located at or near the carboxy terminus, the amino terminus will form the extracellular domain; a TMD at or near the amino terminus will result in the carboxy-terminal portion of the protein forming the extracellular domain; multiple TMDs may be employed to yield a product that has multiple transmembrane domains spanning the membrane.

A second type of membrane attachment moiety comprises a signal that results in the addition of a covalently linked glycolipid. The glycolipid inserts into the lipid bilayer, anchoring the protein to the membrane. This type of membrane attachment moiety also requires a signal sequence for proper targeting to the cell surface. Membrane attachment moieties that direct addition of a glycolipid include a sequence adjacent to the extracellular domain that directs cleavage by an endoprotease, which cleaves off the original membrane attachment moiety and adds a glycolipid such as glycosylphosphatidylinositol (Caras 1991; Abeijon and Hirschberg 1992; Kodukula, Amthauer et al. 1992).

Signal sequences are well known in the art, and can be derived from any protein which is processed for export to the extracellular compartment via the endoplasmic reticulum. Examples of signal sequences include prolactin, VSV-G, and albumin (Rapoport, Curr. Top. Membrane Transport 24:1, 1985).

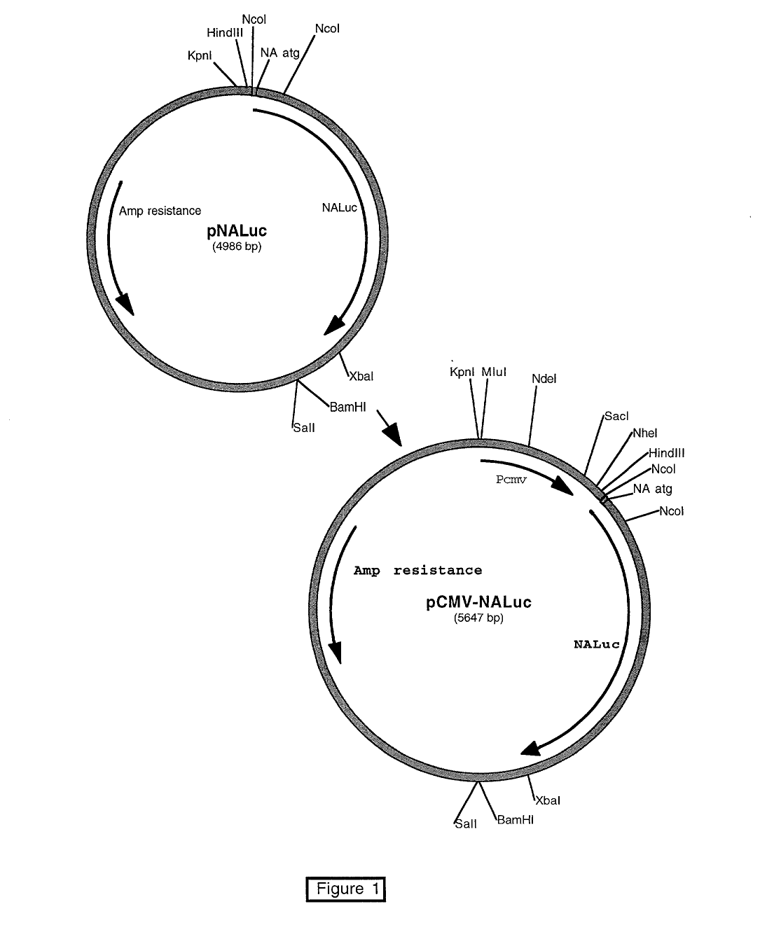

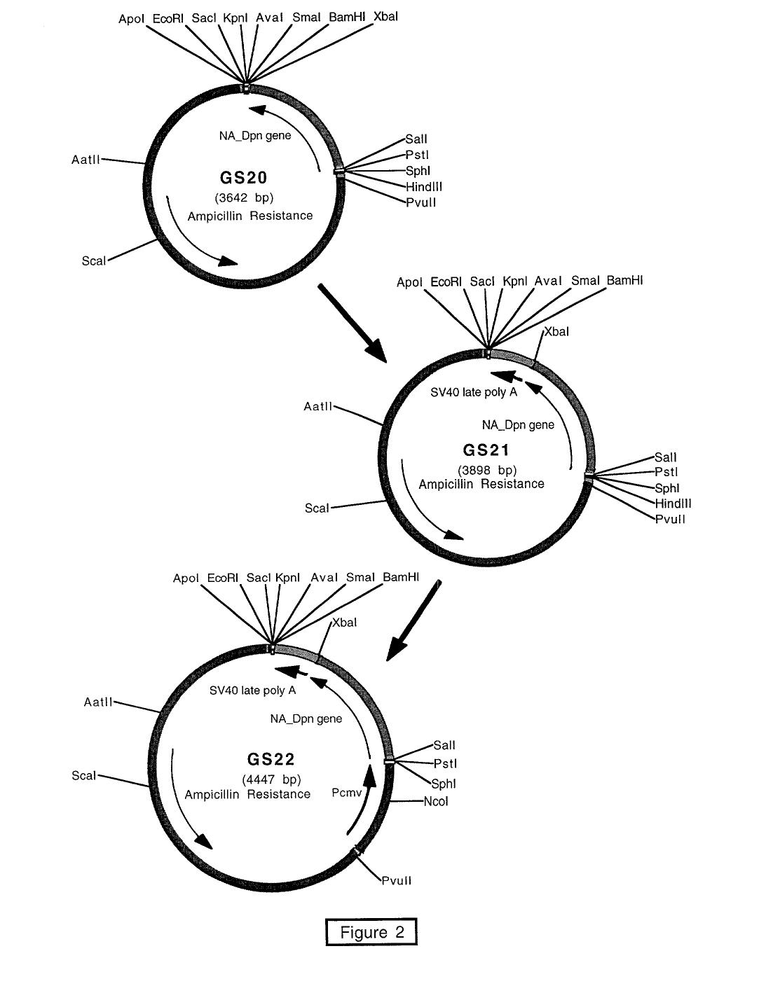

As an example, and as shown in FIG. 1, the coding region of a bacterial luciferase is modified by addition of the first 48 amino acids of the influenza neuraminidase gene (NA) by overlap extension PCR (Current Protocols in Molecular Biology, ed-Ausubel et al.). This modification adds an initiation codon, signal sequence and membrane attachment moiety (transmembrane anchor domain) to the LUC protein sequence. The resulting protein will be transported to the cell surface and anchored to the surface via the transmembrane anchor domain. As another example, shown in FIG. 2, the first 48 amino acids of NA are added to the dpnC gene of Streptococcus pneumoninae that encodes the DpnI restriction endonuclease. The same elements of the NA protein used with the LUC fusion are fused to the amino terminus of DpnI. As a result, the active DpnI molecule will be transported to the cell surface and anchored to the cell surface.

Any of the above described (and herein described) reporter gene constructs (as well as their uses) are encompassed by the invention.

Vectors

The invention also provides vectors useful for detecting and/or quantitating gene expression (for example, assessing activity of an operably linked TRE). Polynucleotides comprising a reporter gene construct may be cloned into any convenient vector, at the discretion of the practitioner. Plasmids, cosmids, yeast artificial chromosomes (YACs), viral vectors, and the like, are contemplated for use in the invention. The vector will comprise a DNA sequence encoding a reporter gene construct as described herein, and may further comprise elements such as selectable markers (operable in either the host cell or in the cells used to clone and produce the vector), origins of replication (operable in either the host cell or in the cells used to clone and produce the vector), multiple cloning site(s) (a DNA sequence of from about 15 to about 150 nucleotides which comprises a plurality of restriction endonuclease sites, preferably wherein the restriction site's only occurrence in the vector is within multiple cloning site), and may further include a TRE, depending on the intended use of the vector. When the vector includes a TRE, it is operably linked to the reporter gene construct. In some embodiments, a TRE is heterologous with respect to the marker and the membrane attachment moiety may or may not be heterologous with respect to the marker.

Expression vectors generally are replicable polynucleotide constructs that contain a polynucleotide encoding a polypeptide of interest. The polynucleotide encoding the polypeptide of interest is operably linked to suitable transcriptional controlling elements, such as promoters, enhancers and terminators. For expression (i.e., translation), one or more translational controlling elements are also usually required, such as ribosome binding sites, translation initiation sites, and stop codons. A polynucleotide sequence encoding a signal peptide can also be included to allow a polypeptide, encoded by an operably linked polynucleotide, to cross and/or lodge in cell membranes or be secreted from the cell. A number of expression vectors suitable for expression in eukaryotic cells including yeast, avian, and mammalian cells are known in the art. Examples of mammalian expression vectors contain both prokaryotic sequence to facilitate the propagation of the vector in bacteria, and one or more eukaryotic transcription units that are expressed in eukaryotic cells. Examples of mammalian expression vectors suitable for transfection of eukaryotic cells include the pcDNAI/amp, pcDNAI/neo, pRc/CMV, pSV2gpt, pSV2neo, pRSVneo, and pHyg derived vectors. Alternatively, derivatives of viruses such as the bovine papilloma virus (BPV-1), or Epstein-Barr virus (pHEB, pREP derived vectors) can be used for expression in mammalian cells. Examples of expression vectors for yeast systems, include YEP24, YIP5, YEP51, YEP52, YES2 and YRPI7, which are cloning and expression vehicles useful for introduction of constructs into S. cerevisiae. Broach et al. (1983) Experimental Manipulation of Gene Expression, ed. M. Inouye, Academic Press. p. 83. Other common vectors, such as YEP13 and the Sikorski series pRS303-306, 313-316, 423-426 can also be used. Vectors pDBV52 and pDBV53 are suitable for expression in C. albicans. Baculovirus expression vectors for expression in insect cells include pVL-derived vectors (such as pVL1392, pVL1393 and pVL941), pAcUW-derived vectors and pBlueBac-derived vectors.

At least two classes of vectors are contemplated. The first class comprises a reporter gene construct operably linked to a TRE of interest. The test vector can then be introduced into host cells to quantitate the activity of the TRE in that host cell type by quantitation of the marker. The TRE may or may not be functional in a cell into which the vector is introduced. Preferably, and especially when gene expression driven by the TRE is to be assessed, the TRE is functional in a host cell into which the vector is introduced. Preferably, the TRE is endogenous with respect to a host cell into which the vector is introduced.

TREs may be isolated using methods standard in the art. For example, a DNA fragment suspected of containing a putative TRE can be tested for function using reporter assays, in which the fragment is linked to a reporter gene. Detection of the expression product indicates presence of a TRE. Alternatively, the fragment could be tested by detecting the presence (and/or amount) of RNA transcript generated by a sequence operably linked to the fragment.

A TRE may or may not be endogenous with respect to the cell in which it is tested for activity using the vectors and methods of this invention. It may also be altered by any of a number of mutations (such as base changes, insertions, deletions) and tested.

A second class of vectors comprise a reporter gene that is not operably linked to a TRE. This second class of vector is intended for use in gene trapping strategies. Typically, the vector is linearized by restriction enzyme digestion at a site 5′ to the reporter gene. Many different types of target cells are useful, including mammalian cells (Hiller, Hengstler et al. 1988), insect cells (Bellen, O'Kane et al. 1989; Wilson, Pearson et al. 1989), and plant cells (Lindsey, Topping et al. 1998). After introduction into the host cells, the vector is integrated into the host cell genome (Macleod, Lovell-Badge et al. 1991; Takeuchi, Yamazaki et al. 1995). Those integration events that result in appropriate positioning of the reporter plus vector backbone adjacent to a promoter will result in expression of the reporter gene from the endogenous promoter (i.e.—promoter trapping). Alternately, the TRE-less vectors may be used to target particular TREs located in the host cell chromosome by homologous recombination. Vectors for use in targeting a particular TRE comprise a sequence located near the TRE (typically a downstream sequence such as coding sequence from the 5′ portion of the gene near the TRE) into which the reporter gene construct is inserted. Homologous recombination of this construct into the host cell chromosome results in an operable linkage between the target TRE and the reporter gene construct. The activity of the TRE can then be measured by quantitating marker present on the cell surface.

In some embodiments, a vector comprises a promoter operably linked to a reporter gene construct, and the manipulations described above (to achieve integration) are performed in order to obtain operable linkage to an enhancer, whose activity may then be measured.

A vector, particularly a vector comprising a TRE operably linked to reporter gene construct, is not limited to a single reporter gene construct. Accordingly, the invention includes vectors containing a second reporter gene construct, both of which may or may not be operably linked to a TRE. As will be apparent to one of skill in the art, when assessment of the activity of more than one TRE in a particular host cell is desired, it may be preferable to assay the activity of multiple TREs in the same cells. This may be accomplished by including different reporter gene constructs, each encoding a different marker and each operably linked to a different TRE, on the same vector. When a vector comprises more than one TRE/reporter gene construct, it is preferred that either the TRE/reporter gene constructs are placed in opposing orientation or are separated by a strong transcriptional stop signal to avoid errors due to read through.

The invention also provides host cells comprising the vectors described herein. These cells are cultured in conventional nutrient media modified as appropriate for inducing TREs, selecting transformants, and/or amplifying the genes encoding the desired sequences.

The cells which are suitable for use in the methods of the present invention with respect to expression, transcriptional control, or for purposes of cloning and propagating a reporter gene construct can be prokaryotic or eukaryotic, but are preferably eukaryotic.

Host systems are known in the art and need not be described in detail herein. Prokaryotic hosts include bacterial cells, for example E. coli, B. subtilis, and mycobacteria. Among eukaryotic hosts are yeast, insect, avian, plant, C. elegans (or nematode) and mammalian cells. Examples of fungi (including yeast) host cells are S. cerevisiae, Kluyveromyces lactis (K lactis), species of Candida including C. albicans and C. glabrata, Aspergillus nidulans, Schizosaccharomyces pombe (S. pombe), Pichia pastoris, and Yarowia lipolytica. Examples of mammalian cells are COS cells, mouse L cells, LNCaP cells, Chinese hamster ovary (CHO) cells, human embryonic kidney (HEK) cells, and African green monkey cells. Xenopus laevis oocytes, or other cells of amphibian origin, may also be used. Mammalian cells may be cell lines (such as those available via the ATCC) or primary cultures. Methods of culturing mammalian cells are known in the art.

Compositions containing cells into which have been introduced vectors described herein are encompassed by this invention.

Marker Detection

The preferred detection reagent for detection of the marker will depend on the identity of the marker. When the marker is an enzyme, the preferred detection reagent is a substrate, whether natural or synthetic, that is detectable after processing by the enzyme. Any type of substrate in which the processed product can be assayed should be suitable, although chromogenic and fluorogenic (e.g., substrates which become colored or fluorescent after enzyme processing) are preferred. Examples of enzyme-substrate combinations are presented in Table 1.

When the marker is a proteinaceous member of a specific binding pair, the marker is preferably detected using the binding partner. As will be apparent to those of skill in the art, binding of the binding partner to the marker can be directly detected (e.g., visualized due to a dye or other modification of the binding partner such as derivatization of the binding partner with an enzyme) under appropriate conditions (e.g., appropriate fluorescence optics for a dye-derivatized binding partner or in the presence of a chromogenic substrate for an enzyme-derivatized binding partner), or is indirectly detected (i.e., the binding partner is detected by addition of a second reagent which binds to or otherwise detects the presence of the binding partner, such as a fluorescently-labeled antibody specific for the binding partner). The binding partner must bind to the marker with sufficiently high affinity to permit detection while bound. Examples of useful binding partners include, but are not limited to, antibodies specific for the marker, non-hydrolyzable analogs of the substrate of a marker enzyme, or naturally occurring ligands of receptors (e.g., biotin for avidin or streptavidin, or the cognate carbohydrate for a lectin).

Methods

The invention provides methods of detecting gene expression using a reporter gene construct, as well as methods for detecting and/or quantitating activity of a TRE and methods for quantitating expression of a marker or marker gene sequence in a host cell.

The methods of the invention utilize a reporter gene construct which is transduced into the host cell of interest. In some embodiments, the reporter gene construct is cloned into a vector in operable linkage with one or more transcriptional regulatory elements (TREs) such as promoters, enhancers, silencers, etc. The resulting synthetic gene is introduced into an appropriate environment to test the elements linked to the reporter gene.

For example, a TRE, or a putative TRE, is obtained on a restriction fragment, or is synthesized using standard methods. The TRE-containing fragment is cloned into an appropriate restriction site (which may arise from a multiple cloning site) in a vector, such that the TRE can regulate transcription of the operably linked sequence encoding the fusion proteins (containing a marker) described herein.

In certain instances it is desirable to test functional elements of genes by inserting the reporter gene into the target genes in their normal chromosomal position by homologous or non-homologous recombination. In these applications the native protein coding sequences of the target gene are replaced or displaced by the reporter gene, functionally linking the reporter gene to the target gene regulatory elements. Alterations in the activity of the target gene elements can be monitored by identifying and/or quantitating the reporter protein.

In addition to the above, essentially any application where a reporter gene is used to monitor, identify and/or quantitate gene expression functional elements could employ the methods of this invention.

The reporter gene vector is introduced into the host cells by any convenient method known to the art. For example, for yeast host cells, the construct may be introduced by electroporation, lithium acetate/PEG and other methods known in the art . Higher eukaryotes may be transformed by electroporation, microprojectile bombardment, calcium phosphate transfection, lipofection, or any other method known to the art. Bacterial host cells may be transfected by electroporation, calcium chloride-mediated transfection, or any other method known in the art.

When the reporter gene vector is one that is intended to incorporate into the host cell's chromosome (i.e., a vector designed to integrate into a chromosomal gene by homologous or non-homologous recombination), the population of host cells which has been transduced is subjected to selection to enrich for those cells which have incorporated the reporter gene construct into the host cell chromosome. Selection is usually carried out after a period of culture in the absence of selection. Selection may be performed on the basis of expression of the reporter gene construct, or it may be on the basis of a separate selectable marker incorporated into the vector. Useful selectable markers include antibiotic resistance genes (e.g., neo^{r}, which confers resistance to the antibiotic G418), genes which complement an auxotrophy of the host cells, and intracellular enzymes heterologous to the host cells (e.g., bacterial β-galactosidase). As is understood in the art, the identity of the selectable marker will depend on the identity of the host cells. Cells transduced with vectors that are not intended for integration into the host cell chromosome are not normally subjected to selection.

The invention also provides methods in which more than one reporter gene construct is used. A plurality (two or more) of reporter gene constructs may be in one vector or more than one vector, which may or may not be extrachromosomal. For example, in one embodiment, one reporter gene construct is on a vector and a second is integrated into a host cell chromosome. In another embodiment, first and second reporter gene constructs are in the same vector. In another embodiment, first and second reporter gene constructs are integrated. The methods encompass use of one or more reporter gene constructs (e.g., two, three, four, five or more).

After a suitable period of incubation (and selection, if appropriate), the cells are cultured under appropriate conditions for expression of the reporter gene construct. Such conditions are generally normal culture conditions appropriate for the particular host cell.

The host cells are assayed for the presence, and optionally for the quantity, of the marker on the surface of the cells. The method of assaying for the presence of the marker will, of course, depend on the identity of the marker. In the case of enzymes (including enzymatically active fragments of enzymes) used as the marker, the marker is preferably assayed by incubating the cells in the presence of a substrate which undergoes a detectable change upon processing by the enzyme (e.g., a chromagenic or fluorogenic substrate). When the marker is a proteinaceous member of a binding pair, the host cells are incubated with the cognate member of the binding pair. Preferably, the added member of the binding pair is labeled so as to render it detectable (e.g., by fluorescent dye, colored particles such as latex beads, or by modification with an enzyme which can be detected), although “indirect” detection of the added member of the binding pair (e.g., by use of a second reagent which detects the presence of the added member of the binding pair) is also contemplated. Enzymes are preferred markers for use in the methods of the invention. A number of enzymes useful as markers, and their cognate detectable substrates, are listed in Table 1.

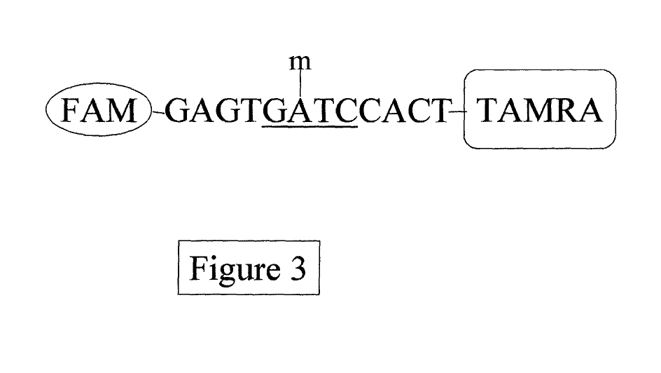

TABLE 1

| Enzyme | Substrate | Mode of Detection |
|---|---|---|
| Beta | O-nitrophenyl-b-D- | Absorbance |
| Galactosidase | pyranogalactoside | |
| | Fluorescein din-b- | Fluorescence |
| | galactopyranoside (FDG) | Chemiluminescence |
| | Galacton (Tropix) | |
| Bacterial | Flavinadenine Dinucleotide/ | Chemilumnescence |
| Luciferase | Decanal | |
| Firefly Luciferase | D-Luciferin | Chemiluminescence |
| DpnI restriction | Self-quenching oligonucleotide | Fluorescence |
| endonuclease | probe containing an internal | |
| | DpnI site (see FIG. 3a) | |
| 5′ Exonuclease | Self-quenching ohigonucleotide | Fluorescence |
| | probe (see FIG. 3b) | |

Generally, the detection reagent is incubated with the host cells under appropriate conditions for signal generation/collection. As will be apparent, the conditions for signal generation/collection will depend on the identity of the detection reagent. For example, when the marker is an enzyme and the detection reagent is a fluorogenic substrate, the cells and detection reagent are incubated under conditions which permit enzymatic activity. The detection reagent signal is collected using an appropriate technology (e.g., fluorimeter for a fluorescent detection reagent, or luminometer for a chemiluminescent detection reagent), dependent on the identity and properties of the detection reagent.

Detection and/or quantitation of the activity of more than one TRE in the same cell(s) is also contemplated. This may be accomplished by using a vector that comprises more than one reporter gene construct (which may or may not be operably linked to a TRE), or by using multiple reporter gene constructs. When multiple TREs are measured in the same cells, the marker is different for each TRE measured, as will be apparent to one of skill in the art. It is preferred that the detection reagents for the different markers do not interfere with each other (e.g., do not fluoresce at the same or similar wavelengths), although it is not required, as the invention provides the ability to perform serial assays without disrupting the cells. Accordingly, measurement of multiple TREs may be accomplished by either adding the appropriate detection markers in combination, followed by simultaneous or serial signal detection, or by serial assay.

Accumulation of fluorescent products of enzymatic reactions may be used to monitor the amount of enzyme present in a reaction or sample. Self-quenching oligonucleotide probes have been developed to monitor DNA amplification by PCR using a 5′ exonuclease assay (Holland et al., PNAS USA 88:7276 (1991); Gelfand et al., U.S. Pat. No. 5,210,015; Lee et al., Nucl. Acids Res. 21:3761 (1993); Livak et al., PCR Methods and Applications 4:357 (1995)). In these methods attachment of a fluorescent reporter molecule to the 5′ end of a single stranded oligonucleotide probe and a molecule capable of quenching this fluorescence at the other end of the oligo results in a substantially non-fluorescent molecule. Release of the fluorescent reporter from the oligo by the 5′ exonuclease activity of a polymerase alleviates the quenching and results in a fluorescent signal.

A similar strategy for converting a non-fluorescent oligonucleotide substrate to a fluorescent product can be employed with endonucleases, in particular, restriction endonucleases (Ghosh, Eis et al. 1994). In this strategy separation of the reporter and quencher molecules is achieved by cleavage of internal phosphodiester bonds by the enzyme.

The substrates for these reactions can include any double stranded DNA molecule which can accommodate 1) attachment of the reporter and quencher molecules in a configuration that results in quenching of the fluorescent signal and 2) includes DNA sequences that can be recognized by the endonuclease. In the example below an oligonucleotide similar to that employed for the 5′ exonuclease assay is designed so that its primary DNA sequence includes a restriction endonuclease recognition site (recognized by DpnI when methylated as shown in FIG. 3, or DpnII when the oligo is not methylated). The oligo is made double stranded by annealing with a complimentary oligo that does not have attached reporter and quencher molecules. Cleavage of the double stranded oligo by DpnI results in two DNA fragments and separates the reporter and quencher molecules, resulting in a fluorescent signal. As with other enzyme assays, accumulation of the fluorescent product will be proportional to enzyme concentration and time.

In some embodiments, the assay for the marker is quantitative. Quantitative assays for the marker are particularly useful when the comparing TREs derived from different individuals, organisms, or genes, or when comparing variations of a TRE, or when making serial measurements. Quantitative assays are well known in the art, and generally involve comparison with a “standard” value. A standard value may be found by utilizing a known quantity of the marker or by utilizing a “control” vector which is transduced into host cells in a manner identical with that used for the “test” vector(s). The control vector normally comprises a TRE which is known to function, preferentially efficiently, in the host cell (e.g., the β-actin promoter in mammalian cells), and is preferably transduced into the host cells at least two different doses (e.g., at different amounts of vector per cell). The control (either the known amount of the marker or the cells transduced by the control vector) is processed using the assay detection system. The assay signal from the control is compared with the assay signal from the test cells. The assay data from the control samples is preferably used to generate a “standard curve”, which may be used to calculate an absolute or relative quantitative value from the assay signal to describe the activity of the test TRE.

Although it is preferred that the control be run contemporaneously with the test samples, it is not required. The control samples may be assayed on a periodic basis (e.g., once each day, week or month) and serve as control for multiple test samples.

As will be apparent to one of skill in the art, the marker expressed from reporter gene constructs may also be used for positive or negative selection, or alternatively isolation, of cells expressing the marker. A number of different affinity-based methods are known in the art for positive and negative selection of cells expressing a particular marker on the cell surface. Positive selection technologies, such as fluorescence-activated cell sorting (FACS), panning, affinity-magnetic separation, and affinity chromatography may be used to physically separate cells expressing a marker from cells not expressing a marker. Positive selection is normally accomplished using a binding partner (e.g., an antibody specific for the marker) either fixed to a solid phase support (i.e., panning or affinity chromatography) or labeled, directly or indirectly (i.e., FACS or affinity-magnetic separation), to facilitate separation of marker-expressing cells from cells which do not express the marker, although non-hydrolyzable substrate analogues may also be useful when the marker is an active enzyme. When desirable, the bound cells may be released from the binding partner or non-hydrolyzable substrate by addition of an excess of soluble marker or by manipulation of pH and/or ionic strength, as is well known in the art.

Selection or isolation of cells expressing a marker(s) as described above may also be a basis for assessing gene expression. For example, gene expression may be detected by isolating the cells expressing marker associated with the cell surface.

Kits

The invention also provides kits which may be used in the methods described herein. The kits comprise at least one container (e.g., a vial or sealed tube) of a vector, along with instructions for use of the vector in detecting and/or quantitating expression of the marker (e.g., TRE activity). The kits may optionally comprise a container of a control vector and/or a detection reagent for detecting the presence of the marker on the host cell surface. The exact composition of the kits will, of course, depend on the intended use of the kit.

In some embodiments, the kit comprises a container of a vector comprising a DNA sequence encoding a reporter gene construct(s) of the invention. The vector may contain a multiple cloning site located near, preferably upstream of the reporter gene construct. A TRE may be easily and simply inserted into such a vector by digesting the vector with a restriction enzyme having a site in the multiple cloning site (MCS), then inserting the TRE.

In other embodiments, the kit comprises a container comprising a vector having two MCS; one located upstream of the reporter gene construct, and one located downstream.

Vectors in this configuration are useful for measuring the activity of TREs located on the host cell chromosome. The two MCS may be used to insert sequences homologous to sequences near, and preferably downstream of, a target TRE.

Optionally, the kits may comprise a control vector. The control vector comprises the same reporter gene construct as the test vectors, and further includes a control TRE operably linked to the reporter gene construct. The control TRE is preferably a TRE that is ubiquitously expressed, such as the β-actin promoter.

The kits may also comprise a detection reagent. As will be apparent to those of skill in the art, the identity of the detection reagent will depend on the identity of the marker. For example, when the marker is β-galactosidase, the kit may also comprise a container of fluorescein din-β-galactopyranoside, a fluorogenic β-galactosidase substrate.

Instructions included with the kit describe the contents of the kit and may describe the use of the vector for detecting/quantitating marker gene expression (such as TRE activity) using the vector. The description of the kit contents will include a description of the structure of the vector, including restriction sites present in the body of the vector and the MCS, as well as any included control vectors and a protocol for using and detecting the detection reagent, if a detection reagent is included in the kit.

The following Examples are intended to illustrate but not limit the invention.

### EXAMPLES

### Example 1

### Construction of Reporter Gene Construct NALuc

A reporter gene construct encoding Firefly luciferase with a signal peptide and a transmembrane domain from influenza virus neuraminidase fused to the amino terminus of the luciferase gene was created. This construct was designated NALuc.

To construct pNALuc, a polynucleotide sequence encoding the first 49 amino acids of influenza virus neuraminidase (NA) plus one linker amino acid (theronine) were inserted into a vector encoding P. pyralis luciferase (Luc) at the 5′ end of the Luc open reading frame (ORF). The resulting vector contains a fusion gene with an ORF that begins with the initiation codon of NA and terminates with the stop codon of Luc.

The first 48 amino acids of NA were PCR amplified from pNA (Brown et al., J. Virol. 62:3824, 1988) using primers NALuc1 (TGT\overline{CCATGG}CATAggcaggagtttaaatgaatc) and NALuc2 (TT\overline{CCATGG}Ttattccagtatggttttgatttc). Note that sequences of the oligos homologous to NA are shown in lower case letters and NcoI restriction sites in the oligos are underlined. The 179 bp fragment of NA containing the SS and TMD of NA (Hiebert and Lamb 1988; Kundu, Jabbar et al. (1991)) was then cleaved with NcoI.

In a parallel reaction, pGL3-basic (Promega, Genbank#U47295) was cleaved with NcoI. Once cleavage was complete, 5′ phosphates were removed from the cleaved pGL3-basic by addition of calf intestinal alkaline phosphatase.

Both the NA fragment and the linear pGL3-basic were removed from the enzyme-containing buffer solution and transferred into water using Qiaquick PCR columns (Qiagen) as directed by the manufacturer.

DNA concentrations were estimated, then the NA fragment and pGL3-basic fragments were mixed at a 1:1 molar ratio and ligated using T4 DNA ligase at 15° C. for 18 hours. Following transformation of E. coli host cells, clones with the desired structure were identified by restriction enzyme digestion and gel electrophoresis.

### Example 2

### Construction of a Vector with a Heterologous TRE Driving Transcription of NALuc

The immediate early enhancer and promoter from cytomegalovirus (CMV; Foecking and Hofstetter Gene 45:101, 9185) was inserted into pNALuc in the correct orientation to direct transcription of NALuc.

The 657 base pair pCMV fragment was released from pcDNA3.1 (Invitrogen) using MluI plus NheI, purified by gel electrophoresis, then ligated to similarly cut pNALuc. Desired clones were identified from E. coli transformants by restriction digestion.

### Example 3

### Detection and Quantitation of PCMV TRE Activity in COS-7 Cells

Cos-7 cells (Gluzman Y. Cell 23: 175-182, 1981) were transfected with one of the following plasmids: pNALuc, pCMV-NALuc clone 2, or pCMV-NALuc clone 9. Clones 2 and 9 were independent isolates from the same experiment.

The cells were plated at 500,000 cells per well in 6 well plates, and each well was transfected with 0.5 mg of one of the above plasmid DNAs. Mock transfected cells were included as a negative control. Transfections were performed with Fugene6 transfection reagent (Roche) according to the manufacturer's instructions. Following transfection the cells were incubated in growth medium for two days.

To assay luciferase activity in the transfected cells, the cells were scraped off of the plastic substrate in 0.5 ml of phosphate buffered saline. Following brief centrifugation, the supernatant was removed and the cell pellet was resuspended in 100 μl phosphate buffered saline. Ten microliters of cell suspension was added to a luminometer cuvette, followed by 100 μl of luciferin assay reagent (Promega). The sample was mixed and luminescence measured for 10 seconds using an Analytical Luminescence Monolight 2010 luminometer.

Mock transfected cells yielded a background luminescence reading of 92 relative light units (RLU) (Table 2). Lysates from cells transfected with pNALuc (which lacks TRE's driving NALuc expression) yielded four times more luminescence than mock transfected cells, indicating a low level of transcription of pNALuc from cryptic promoters in the vector. Lysates from cells transfected with pCMV-NALuc clone 2 and pCMV-NALuc clone 9 yielded luminescence 2900-fold and 3 100-fold above background, respectively. These results demonstrate that the NALuc gene in pNALuc and pCMV-NALuc is highly active for its cognate substrate. In addition, the experiment shows that NALuc can be used as a quantitative indicator of transcription in intact cells.

TABLE 2

| Plasmid | Relative Light Units | Fold Induction |
|---|---|---|
| Mock transfected | 92 | 1 |
| pNALuc clone 2 | 367 | 4 |
| pCMV-NALuc clone 2 | 268087 | 2913 |
| pCMV-NALuc clone 9 | 281504 | 3060 |

### Example 4

### Construction of Reporter Gene Construct GS20 and GS21

Overlap extension PCR, similar to that used in Example 1, was used to fuse the DNA sequences encoding the first 48 amino acids of influenza virus neuraminidase with the dpnC gene of S. pneumoniae that encodes the DpnI restriction endonuclease (Lacks et al., Cell 46:993, 1986).

The dpnC gene from pLS252 (ATCC#67494) was PCR amplified using primers Oligo 5 (5′-TGTCCCATCCGTGGGatgatgaagcgtgtg-3′, bases 1-15 are from the neuraminidase gene (CAPS) and bases 16-30 are from the 5′ end of the dpnC open reading frame (ORF), italics), Oligo 6 (5′-gtt\overline{tctaga}tcataatttccgata-3′, containing a XbaI site (underlined) and sequences from the 3′ end of the dpnC ORF (italics)). The first 164 bases of the influenza neuraminidase gene were PCR amplified using primers Oligo 7 (5′-tgt\overline{gtcgac}TAATCTCAATATGGA-3′, containing an Sal I site (underlined) and sequences from −10 to −15 5 relative to the first base of the initiation codon of the neuramindase gene (CAPS)) and Oligo 8 (5′-cacacgcttcatcatCCCACGGATGGGACA-3′, which is the complement of Oligo 5).

The dpnC and neuraminidase PCR products were purified using Qiagen Qiaquick PCR purification kit, then one microliter of each was added to 98 μl of water. One microliter of this template mixture was amplified by PCR with Oligos 6 and 7. The resulting 776 bp fusion fragment of the NA amino terminus with dpnC was gel purified from the two small, non-fused fragments.

The fusion DNA fragment was cut with XbaI and SalI, then ligated to similarly cut pGEM4 (Genbank#VB0039) to construct GS20 (FIG. 2).

For applications in which a polyadenylation signal is required for reporter expression, the polyadenylation signal from the SV40 small t antigen derived as an XbaI/BamHI fragment from pGL3-basic was inserted between the XbaI and BamHI sites of GS20 to construct GS21 (FIG. 2).

### Example 5

### Fluorescent Assay for Restriction Endonuclease Activity

A fluorogenic substrate oligonucleotide (Ghosh, Eis et al. 1994) containing the recognition sequence for DpnII (FIG. 3) was treated with DNAseI, EcoRI or DpnII as an example of an assay to specifically detect cell surface expression of NADpn from example 4.

Covalent attachment of TAMRA at the 3′ end of the oligo quenches fluorescence emission from the FAM dye molecule covalently attached to the 5′ end of the oligo through fluorescence resonance energy transfer (FRET). A DpnII site is formed by annealing the labeled oligo to a non-labeled, complimentary strand. Recognition of the DpnII site between the dye molecules by DpnII or NADpn will be followed by double strand cleavage and separation of the fluor and quencher dyes. FRET is reduced by separation of the dyes and can be quantitatively detected as an increase in fluorescence of the reaction. Reactions containing 0.36 μg of Dpn oligo, 1 μg/ml of bovine serum albumin, 0.1 μg/ml E. coli genomic DNA and appropriate restriction enzyme buffer in 20 μl were assembled at 0° C. in an ABI Prism 7700 Sequence Detection Instrument. Twenty microliters of buffer containing BSA, E. coli genomic DNA and one of the following were added to appropriate wells:

EcoRI (20U)

DpnII (1 OU)

DNAse I (100U)

No enzyme

The temperature was raised to 37° C. and the fluorescence of the FAM dye in each reaction was measured every 4 seconds. The data are plotted as fluorescence intensity versus time.

In panel A, the probe was incubated in the absence of enzyme, demonstrating the lack of increased fluorescence in the absence of added nuclease. In panel B DNAse I was added to the probe to verify that an increase in absorbance would be seen upon degradation of the probe. Immediately shift of temperature to 37° C., an increase in fluorescence was seen which reached a plateau approximately ⅓ of the way through the time course (about 20 minutes).

The specificity of the assay was demonstrated by the lack of increase in fluorescence intensity upon addition of EcoRI (panel C). In contrast, the reaction containing DpnII exhibited an increase in fluorescence intensity similar to that seen in the DNAse I reaction (panel D), reaching a plateau approximately ½ of the way through the experiment.

### Example 6

### Construction of a Bacterial Luciferase Reporter Gene Construct

Overlap extension PCR (Current Protocols in Molecular Biology) was used to fuse the DNA sequences encoding the first 48 amino acids of influenza virus neuraminidase with a monocistronic bacterial luciferase gene from V. harveyi (Boylan et al., J. Biol. Chem. 264:1915 (1989)).

The LuxAB gene from pT7-mut3 (Boylan et al., J. Biol. Chem. 264:1915 (1989) was PCR amplified using primers Oligo 1 (5′-TGTCCCATCCGTGGGatgaaatttggaaac-3′, Bases 1-15 are from the neuraminidase gene (CAPS) and bases 16-30 are from the 5′ end of the LuxAB open reading frame (ORF; italics)) and Oligo 2 (5′-gtt\overline{tctaga}ttacgagtggtatttg-3′, containing a XbaI site (underlined) and sequences from the 3 end of the 5 LuxAB ORF (italics)). The first 164 bases of the influenza neuraminidase gene were PCR amplified using primers Oligo 3 (5′-tgt\overline{gtcgac}TAATCTCAATATGGA-3′, containing a SalI site (underlined) and sequences from −10 to −15 relative to the first base of the initiation codon of the neuraminidase gene (CAPS)) and Oligo 4 (5′-gtttccaaatttcatCCCACGGATGGGACA-3′, which is the complement of Oligo 1).

The PCR products were purified using Qiagen Qiaquick PCR purification kit, then one microliter of each was added to 98 μl of water. One microliter of this template mixture was amplified by PCR with Oligos 2 and 3. The resulting 2327 bp fusion fragment of the NA amino terminus with LuxAB was gel purified from the two small, non-fused fragments.

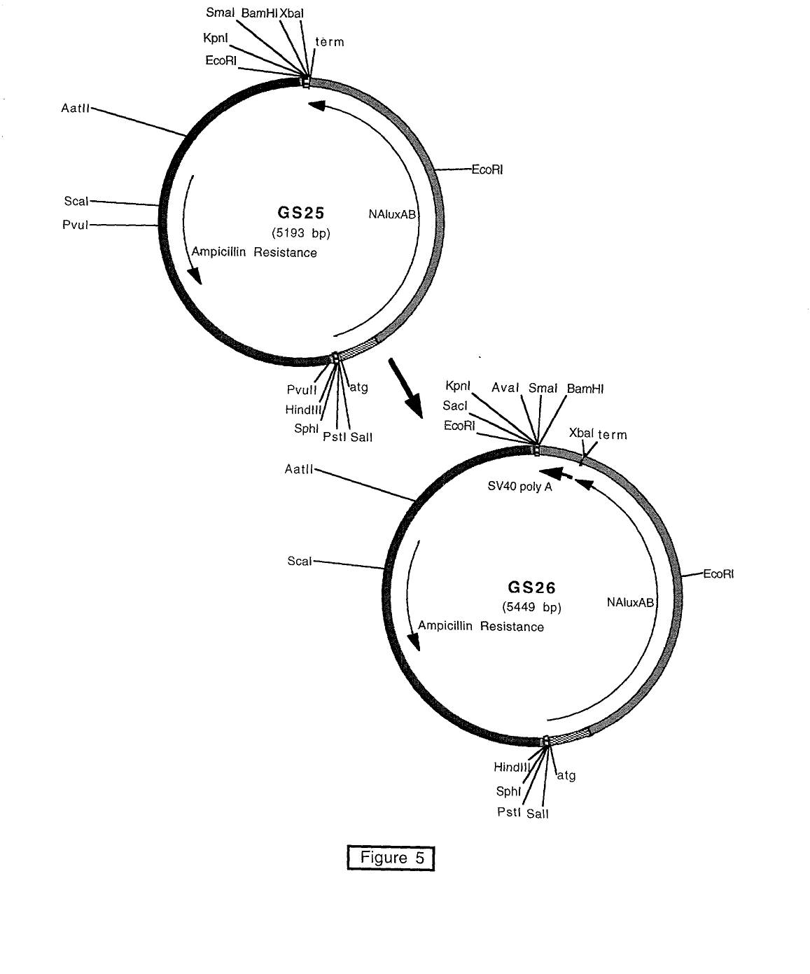

The fusion DNA fragment was cut with XbaI and SaIl, then ligated to similarly cut pGEM4 (Genbank#VB0039) to construct GS25 (FIG. 5).

For applications in which a polyadenylation signal is required for reporter expression, the polyadenlylation signal from the SV4O small t antigen derived as an XbaI/BamHI fragment from pGL3-basic was inserted between the XbaI and BamIlI sites of GS25 to construct GS26 (FIG. 5).

### Example 7

### Assay of TRE Activity Assay Using DpnI as a Marker

The function of the DpnI marker was tested by performing a dose-response experiment in MCF-7 breast cancer cells transiently transfected with a constitutively expressed NADpn gene.

The constitutively expressed NADpnI vector was constructed from GS21 by ligating the SV40 early promoter/enhancer into the HinDIII site of GS21. The cytomegalovirus immediate early promoter/enhancer (Pcmv) was PCR amplified from pCMVbeta (Genbank Accession #U02451) using primers Oligo 9 5′-gtg\overline{aagctt}GAGCTTGCATGCCTG-3′, HinDIII site underlined, bases homologous to the S′ end of the Pcmv promoter are capitalized) and Oligo 10 (5′-tta\overline{aagctt}ACGGTTCACTAAACG-3′, HinDIII site underlined, bases homologous to the 3′ end of the Pcmv promoter are capitalized). The 540 bp PCR product was cut with HindIll, then ligated into similarly cut GS21 to construct GS22 (FIG. 2).

Aliquots of 10 million exponentially growing MCF-7 cells were transfected by electroporation using conventional conditions (Current Protocols in Molecular Biology) with variable amounts of GS22 as described in table 3. After electroporation, cells were plated in three 10 cm dishes in 10 ml growth medium at 37° C. for 48 hours.

To assay cell surface DpnI activity, 20 μg of substrate (see FIG. 3 and Example 4) in phenol red free DMEM plus 10% fetal bovine serum was addled to each dish at time zero. At each time point one ml aliquots of medium were removed from each dish and the fluorescence intensity measured. Data were plotted as fluorescence intensity vs. time for each amount of GS22 transfected.

TABLE 3
Transfection of MCF-7 cells with GS22.

| Sample Number | DNA (μg) |
|---|---|
| 1 | 1 |
| 2 | 10 |
| 3 | 50 |

### REFERENCES (NON-INCLUSIVE)

Abeijon, C. and C. B. Hirschberg (1992). “Topography of glycosylation reactions in the endoplasmic reticulum.” Trends Biochem Sci 17(1): 32-6.

Bellen, H. J., C. J. O'Kane, et al. (1989). “P-element-mediated enhancer detection: a versatile method to study development in Drosophila.” Genes Dev 3(9): 1288-300.

Brown, D. J., B. G. Hogue, et al. (1988). “Redundancy of signal and anchor functions in the NH2-terminal uncharged region of influenza virus neuraminidase, a class II membrane glycoprotein.” J Virol 62(10): 3824-31.

Caras, I. W. (1991). “An internally positioned signal can direct attachment of a glycophospholipid membrane anchor.” J Cell Biol 113(1): 77-85.

Ghosh, S. S., P. S. Eis, et al. (1994). “Real time kinetics of restriction endonuclease cleavage monitored by fluorescence resonance energy transfer.” Nucleic Acids Res 22(15): 3155-9.

Hegde, R. S. and V. R. Lingappa (1997). “Membrane protein biogenesis: regulated complexity at the endoplasmic reticulum.” Cell 91(5): 575-82.

Hiebert, S. W. and R. A. Lamb (1988). “Cell surface expression of glycosylated, nonglycosylated, and truncated forms of a cytoplasmic protein pyruvate kinase.” J Cell Biol 107(3): 865-76.

Hiller, S., M. Hengstler, et al. (1988). “Insertional activation of a promoterless thymidine kinase gene.” Mol Cell Biol 8(8): 3298-302.

Kalderon, D., B. L. Roberts, et al. (1984). “A short amino acid sequence able to specify nuclear location.” Cell 39(3 Pt 2): 499-509.

Kodukula, K., R. Amthauer, et al. (1992). “Biosynthesis of phosphatidylinositolglycan (PI-G)-anchored membrane proteins in cell-free systems: PI-G is an obligatory cosubstrate for COOH-terminal processing of nascent proteins.” Proc Natl Acad Sci USA 89(11): 4982-5.

Kundu, A., M. A. Jabbar, et al. (1991). “Cell surface transport, oligomerization, and endocytosis of chimeric type II glycoproteins: role of cytoplasmic and anchor domains.” Mol Cell Biol 11(5): 2675-85.

Lindsey, K., J. F. Topping, et al. (1998). “Dissecting embryonic and seedling morphogenesis in Arabidopsis by promoter trap insertional mutagenesis.” Symp Soc Exp Biol 51: 1-10.

Macleod, D., R. Lovell-Badge, et al. (1991). “A promoter trap in embryonic stem (ES) cells selects for integration of DNA into CpG islands.” Nucleic Acids Res 19(1): 17-23.

Palade, G. (1975). “Intracellular aspects of the process of protein synthesis.” Science 189(4200): 347-58.

Takeuchi, T., Y. Yamazaki, et al. (1995). “Gene trap capture of a novel mouse gene, jumonji, required for neural tube formation.” Genes Dev 9(10): 1211-22.

van Geest, M. and J. S. Lolkema (2000). “Membrane topology and insertion of membrane proteins: search for topogenic signals.” Microbiol Mol Biol Rev 64(1): 13-33.

Wickner, W. T. and H. F. Lodish (1985). “Multiple mechanisms of protein insertion into and across membranes.” Science 230(4724): 400-7.

Wilson, C., R. K. Pearson, et al. (1989). “P-element-mediated enhancer detection: an efficient method for isolating and characterizing developmentally regulated genes in Drosophila.” Genes Dev 3(9): 1301-13.

Although the foregoing invention has been described in detail by way of illustration and example for purposes of clarity of understanding, it will be apparent to those skilled in the art that certain changes and modifications can be practiced. Therefore, the description and examples should not be construed as limiting the scope of the invention, which is delineated by the appended claims.

1. A vector useful for expressing a marker on a cell surface, comprising a nucleotide sequence encoding a fusion polypeptide, said fusion polypeptide comprising (a) a signal sequence; (b) a membrane attachment moiety; and (c) a marker, wherein said signal sequence, membrane attachment moiety and marker are operably linked in frame and wherein said vector lacks a transcriptional regulatory element (TRE) operably linked with said nucleotide sequence.

2. The vector of claim 1, wherein the membrane attachment moiety is a transmembrane domain.

3. The vector of any preceding claim, further comprising a nucleotide sequence for selection in mammalian cells.

4. The vector of any of claims 1-3, wherein the marker is an enzyme.

5. The vector of claim 4, wherein the enzyme is a restriction endonuclease.

6. The vector of any of claims 1-3, wherein the marker is a domain of an enzyme.

7. The vector of any of claims 1-3, wherein the marker is a subunit of an enzyme.

8. The vector of any of claims 1-3, wherein the marker is a proteinaceous member of a binding pair.

9. The vector of any of claims 1-3, wherein the marker is an epitope.

10. The vector of any of claims 1-3, further comprising a multiple cloning site.

11. A host cell comprising the vector of any of claims 1-3.

12. The host cell of claim 11, which is mammalian.

13. A kit comprising the vector of any of claims 1-3.

14. The kit of claim 13, further comprising a detection reagent.

15. A method for detecting expression of a reporter gene construct in a host cell, comprising:
detecting a marker encoded by the reporter gene construct, wherein the marker is associated with the cell surface, and wherein said reporter gene construct comprises a nucleotide sequence encoding a fusion polypeptide comprising a signal sequence, a membrane attachment moiety and a marker, said membrane attachment moiety heterologous to said marker, and wherein said signal sequence, membrane attachment moiety and marker are operably linked in frame, and wherein said nucleotide sequence is operably linked to a transcriptional response element (TRE) which is functional in said host cell.

16. The method of claim 15, wherein the TRE is endogenous with respect to the host cell.

17. The method of claim 15, wherein the marker is not naturally associated with the host cell surface.

18. The method of claim 15, wherein the reporter gene construct is in a vector which is extrachromosomal.

19. The method of claim 15, wherein the reporter gene construct is integrated into a host cell chromosome.

20. The method of claim 15, wherein the marker is an enzyme.

21. The method of claim 20, wherein the enzyme is a restriction endonuclease.

22. The method of claim 15, wherein the marker is a domain of an enzyme.

23. The method of claim 15, wherein the marker is a subunit of an enzyme.

24. The method of claim 15, wherein the marker is a proteinaceous member of a binding pair.

25. A method of isolating a cell which expresses a marker on its surface, said marker expressed from a reporter gene construct comprising a nucleotide sequence encoding a fusion polypeptide comprising a signal sequence, a membrane attachment moiety and a marker, said membrane attachment moiety heterologous with respect to said marker, and wherein said signal sequence, membrane attachment moiety and marker are operably linked in frame, and wherein said nucleotide sequence is operably linked to a transcriptional response element (TRE) which is functional in said host cell, said method comprising
binding the marker to a binding partner which specifically binds to the marker to form a complex between the binding partner and the marker on the cell surface; and isolating the cells which contain the complex.

26. A method of detecting expression of a reporter gene construct encoding a marker which is associated with the cell surface, comprising:
binding the marker to a binding partner which specifically binds to the marker to form a complex between the binding partner and the marker on the cell surface; and isolating the cells which contain the complex, wherein said marker is expressed from a reporter gene construct integrated into a chromosome of the host cell, said reporter gene construct comprising a nucleotide sequence encoding a fusion polypeptide comprising a signal sequence, a membrane attachment moiety and a marker, said membrane attachment moiety heterologous with respect to said marker, and wherein said signal sequence, membrane attachment moiety and marker are operably linked in frame, and wherein said nucleotide sequence is operably linked to a transcriptional response element (TRE) which is functional in said host cell.

27. A method for detecting expression of a reporter gene construct in a host cell, comprising:
detecting a marker encoded by the reporter gene construct, wherein the marker is associated with the cell surface, and wherein said reporter gene construct comprises a nucleotide sequence encoding a fusion polypeptide comprising a signal sequence, a membrane attachment moiety and a marker, and wherein said signal sequence, membrane attachment moiety and marker are operably linked in frame, and wherein said nucleotide sequence is operably linked to a transcriptional response element (TRE) which is functional in said host cell and wherein the TRE is heterologous with respect to the marker.
